(19) United States
(12) Patent Application Publication
(10) Pub. No.: US 2012/0226291 A1
(43) Pub. Date: Sep. 6, 2012

(54) Surgical Ligation Clip and Applicator Device

(75) Inventors: Anthony A. Malizia, Atlanta, GA (US); Elizabeth L. Malizia, Atlanta, GA (US); Brian VanHiel, Smyrna, GA (US); Kirk Charles, Austell, GA (US); Maureen Carroll, Atlanta, GA (US); Franklin Bost, Newnan, GA (US)

(73) Assignee: JENKINS CLINIC, INC., Atlanta, GA (US)

(21) Appl. No.: 13/410,703

(22) Filed: Mar. 2, 2012

Related U.S. Application Data
(60) Provisional application No. 61/449,247, filed on Mar. 4, 2011.

(51) Int. Cl. A61B 17/10; A61B 17/03

(52) U.S. Cl. 606/143; 606/151

(57) ABSTRACT

A surgical tissue ligation clip and an applicator device for delivery of multiple ligation clips is provided. The surgical ligation clips have first and second leg members joined by a resilient hinge at one end and a deflectable hook member and a locking element on the opposite end. The surgical tissue ligation clip has a proximal tissue shield that prevents tissue from being pinched at the hinge, and/or a distal tissue shield that prevents tissue from being pinched between the hook member and locking element and interfering with securely closing the ligation clip for implantation. A minimally invasive applicator for the surgical ligation clip is provided for delivering individually arranged ligation clips in line, incising target tissue and delivering a plurality of surgical ligation clips at the tissue site.

### CROSS REFERENCE TO RELATED APPLICATION

This application claims the priority benefit of U.S. Provisional Patent Application No. 61/449,247 filed on Mar. 4, 2011. The contents of this application is hereby incorporated by reference in its entirety.

### FIELD OF THE INVENTION

The present invention relates generally to vessel sealing devices, and particularly to a system, apparatus, and methods for providing a surgical clip device for ligating tissues during surgical procedures.

### BACKGROUND OF THE INVENTION

A radical prostatectomy is frequently the recommended procedure for treating cancerous prostate tissue. The primary blood supply for the prostate are the prostatic pedicles. In the process of removing the prostate, the pedicles must be ligated and divided. Careful hemostasis of the prostatic vascular pedicles is required to avoid collateral injury to the prostate and adjacent neural tissue, which is necessary to preserve continence and potency after recovery, and to create negative surgical margins to insure complete removal of cancerous tissue.

Currently available surgical ligation clips and delivery means are not ideally adapted for use on prostatic vascular pedicles. The pedicle tissue is considerably more dense than most other vascularized connective tissues. The lateral prostate pedicle is also thicker in size than the underlying neurovascular bundle. Therefore, the forces required to close the ligation clip around the prostate pedicle tissue are much greater than for most other surgical ligation clip applications.

Furthermore, the bundling of excess tissue associated with the thick pedicle vasculature around the ligation clip hinge or the ligation clip closure mechanism frequently causes currently available clips to fail through breakage or lack of secure engagement. Existing clips that have sharpened features disposed thereon for tissue penetration also present a disadvantageous risk of unintended laceration after being secured onto target tissues. Existing systems for ligation clip placement also inadequately protect ancillary tissue laceration not only after being secured to the target tissue, but upon delivery after exiting the trocar or other laproscopic delivery and applicator devices. Existing clips also frequently fail to remain securely engaged within the applicator tool during delivery or placement and can misfire or inadvertently fall into the surgical field.

Therefore, there is a need in the art for improved surgical ligation clips and applicator devices, for use in open, laproscopic, and robotic surgeries, and in particular for those specifically designed for use in ligating pedicles such as in a prostatectomy, hysterectomy, a bowel resection, and similar procedures.

### SUMMARY OF THE INVENTION

The present invention provides a surgical tissue ligation clip and an applicator device for delivery of single ligation clips or multiple ligation clips. The surgical ligation clips each have first and second leg members each having a central portion, a proximal end, a distal end, a tissue clamping inner surface, and an opposite outer surface with elongated sides. The leg members are joined at their proximal ends by a resilient hinge such that the tissue clamping inner surface of one leg member is in movable opposition to the tissue clamping inner surface of the opposite leg member.

In certain embodiments, the surgical tissue ligation clip further has a deflectable hook member on the distal end of the first leg member curved proximally toward said second leg member, and a locking element on the distal end of the second leg member in a position complementary to said hook member. When the first and second leg members are moved from an open position to a closed position about the hinge, the hook member deflects about the locking element to lock the clip in a closed position.

The surgical tissue ligation clip of the present invention further has a proximal tissue shield extending inwardly from the proximal portion of the first or second leg members past the tissue clamping inner surface of the opposite leg member when the leg members are in a partially closed position. The proximal tissue shield prevents tissue that is being clamped between the inner surfaces of the leg members from being pinched and cut at the hinge to avoid undesired hemorrhaging, disassociating the hinge, or preventing closure of the ligation clip for implantation.

The surgical tissue ligation clip can further have a distal tissue shield extending outwardly from side to side of the distal portion of the outer surface of the second leg member proximal to locking element. The distal tissue shield prevents tissue being clamped between the inner surfaces of the leg members from being pinched between the hook member and locking element to avoid undesired hemorrhaging, interfering with securely closing the ligation clip for implantation, or disassociating the hinge. Tissue shielding and clearing away from the ligation clip mechanism also provides improved tactile feedback to the user when the clip is closed.

The present invention further provides a minimally invasive applicator for a surgical ligation clip. The applicator can be configured for single clip delivery, or be adapted for receiving a plurality of surgical ligation clips individually arranged in line for movement therethrough. The elongated surgical ligation clip chamber has a proximal end, a central storage region and an open distal end. The surgical ligation clip applicator further has a pair of jaws disposed on the open distal end of the chamber having a sharpened incising tooth member extending from at least one side towards the opposite jaw. The jaws selectively articulate between an open position for retaining an open ligation clip therebetween, and a closed position for penetrating interfering tissue with the sharpened incising tooth member between an opposing pair of jaws, and securely closing the ligation clip onto the target tissue.

The applicator for a surgical ligation clip further has a handle disposed on the proximal end of the elongated chamber for user engagement. The applicator further has a jaw actuator mechanism within the elongated chamber comprising a movable actuator rod in responsive communication with the handle to open and close the jaws. The applicator further has a ligation dip advancement mechanism within the elongated chamber in responsive communication with the handle for selectively indexing clips distally one at a time into the jaws of the applicator.

The invention provides further embodiments and alternatives to the above ligation clip and applicator, in addition to methods of using and methods of manufacturing the same.

### BRIEF DESCRIPTION OF THE DRAWINGS

Reference will now be made to the accompanying drawings, which are not necessarily drawn to scale, and wherein:

FIG. 1A illustrates an upper distal perspective view of an exemplary ligation clip in an open position in accordance with one embodiment of the invention.

FIG. 1B illustrates an upper proximal perspective view of an exemplary ligation clip in an open position in accordance with one embodiment of the invention.

FIG. 1C illustrates an lower distal perspective view of an exemplary ligation clip in an open position in accordance with one embodiment of the invention.

FIG. 1D illustrates an lower proximal perspective view of an exemplary ligation clip in an open position in accordance with one embodiment of the invention.

FIG. 1E illustrates a distal end view of an exemplary ligation clip in an open position in accordance with one embodiment of the invention.

FIG. 1F illustrates a side cross-section view of an exemplary ligation clip in an open position in accordance with one embodiment of the invention.

FIG. 2A illustrates an upper distal perspective view of an exemplary ligation clip in a partially closed position in accordance with one embodiment of the invention.

FIG. 2B illustrates an upper proximal perspective view of an exemplary ligation clip in a partially closed position in accordance with one embodiment of the invention.

FIG. 2C illustrates an lower distal perspective view of an exemplary ligation clip in a partially closed position in accordance with one embodiment of the invention.

FIG. 2D illustrates an lower proximal perspective view of an exemplary ligation clip in a partially closed position in accordance with one embodiment of the invention.

FIG. 2E illustrates a distal end view of an exemplary ligation clip in a partially closed position in accordance with one embodiment of the invention.

FIG. 2F illustrates a side cross-section view of an exemplary ligation clip in a partially closed position in accordance with one embodiment of the invention:

FIG. 3A illustrates an upper distal perspective view of an exemplary ligation clip in a closed position in accordance with one embodiment of the invention.

FIG. 3B illustrates an upper proximal perspective view of an exemplary ligation clip in a closed position in accordance with one embodiment of the invention.

FIG. 3C illustrates an lower distal perspective view of an exemplary ligation clip in an closed position in accordance with one embodiment of the invention.

FIG. 3D illustrates an lower proximal perspective view of an exemplary ligation clip in a closed position in accordance with one embodiment of the invention.

FIG. 3E illustrates a distal end view of an exemplary ligation clip in a closed position in accordance with one embodiment of the invention.

FIG. 3F illustrates a side cross-section view of an exemplary ligation clip in a closed position in accordance with one embodiment of the invention.

FIG. 3G illustrates a proximal end view of an exemplary ligation clip in a closed position in accordance with one embodiment of the invention.

FIG. 4A illustrates an upper distal perspective view of an exemplary ligation clip in an open position in accordance with one embodiment of the invention.

FIG. 4B illustrates a side view of an exemplary ligation clip in an open position in accordance with one embodiment of the invention.

FIG. 4C illustrates a side view of an exemplary ligation clip in a closed position in accordance with one embodiment of the invention.

FIG. 5 illustrates an upper distal perspective view of an exemplary ligation clip applicator in accordance with one embodiment of the invention.

FIG. 6A illustrates an overhead view of an exemplary ligation clip applicator in accordance with one embodiment of the invention.

FIG. 6B illustrates a side view of an exemplary ligation clip applicator in accordance with one embodiment of the invention.

FIG. 6C illustrates a lower distal perspective view of an exemplary ligation clip applicator in accordance with one embodiment of the invention.

FIG. 7A illustrates an upper distal perspective detail view of an exemplary ligation clip applicator in accordance with one embodiment of the invention.

FIG. 7B illustrates an upper distal perspective detail view of advancing the ligation clips in the applicator in accordance with one embodiment of the invention.

FIG. 7C illustrates an upper distal perspective detail view of advancing the ligation clips further in the applicator in accordance with one embodiment of the invention.

FIG. 7D illustrates an upper distal perspective detail view of advancing the ligation clips in the applicator with the distal most ligation clip registering in the jaws in accordance with one embodiment of the invention.

FIG. 8A illustrates a side cross-section detail view of an exemplary ligation clip applicator in the open position loaded with a ligation clip in accordance with one embodiment of the invention.

FIG. 8B illustrates a side cross-section detail view of an exemplary ligation clip applicator in the partially closed position loaded with a ligation clip in accordance with one embodiment of the invention.

FIG. 8C illustrates a side cross-section detail view of an exemplary ligation clip applicator in the closed position loaded with a ligation clip in accordance with one embodiment of the invention.

### DETAILED DESCRIPTION OF THE INVENTION

Embodiments of the invention now will be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. The singular forms “a,” “an,” and “the” can refer to plural instances unless context clearly dictates otherwise or unless explicitly stated.

The various embodiments described herein provide exemplary devices, systems, methods of use and manufacture of surgical ligation clips and applicators for patient tissue in need thereof. The present invention provides a surgical tissue ligation clip, comprising first and second leg members each having a central portion, a proximal end, a distal end, a tissue clamping inner surface, and an opposite outer surface with elongated sides. The leg members are joined at their proximal ends by a resilient hinge such that the tissue clamping inner surface of one leg member is in movable opposition to the tissue clamping inner surface of the other leg member.

The surgical tissue ligation clip further comprises a deflectable hook member on the distal end of the first leg member curved proximally toward said second leg member, and a locking element on the distal end of the second leg member in a position complementary to said hook member. When the first and second leg members are moved from an open position to a closed position about the hinge, the hook member deflects about the locking element to lock the clip in a closed position. The invention contemplates the use of any latching or locking mechanism, particularly those that can be selectively disengaged for removal, as are well known in the art.

The surgical tissue ligation clip further comprises a proximal tissue shield. The proximal tissue shield can extend inwardly from the proximal portion of the first or second leg members past the tissue clamping inner surface of the opposite leg member when the leg members are in a partially closed position. The proximal tissue shield prevents tissue that is being clamped or ligated between the inner surfaces of the leg members from being pinched at the hinge. The proximal tissue shield prevents unintended acute tissue perforation and hemorrhaging, as well as hinge failure due to excessive pressure loading.

The surgical tissue ligation clip can further comprise a distal tissue shield extending outwardly from side to side of the distal portion of the outer surface of the second leg member proximal to locking element. The distal tissue shield prevents tissue being clamped between the inner surfaces of the leg members from being pinched between the hook member and locking element to avoid undesired tissue perforation and hemorrhaging, as well as avoiding interference with securely closing the ligation clip for implantation due to excessive pressure loading at the engagement mechanism, or disassociating the hinge, as well as to improve operator feedback of the tactile sensation of correct clip closure. Once the clip is closed and secure, the tissue shield also prevents further disturbances by surrounding tissue or anatomical structures against displacement of the locking mechanism.

In certain embodiments of the surgical ligation clip, the second leg member defines a centrally located elongated slot into which the central portion of the first leg member passes when the ligation clip is in a closed position. The tissue clamping inner surface of the second leg member has two surfaces which are in movable opposition to, but not in contact with, the inner surface of the first leg member when the ligation clip is in a closed position.

In certain embodiments of the surgical ligation clip, closing the ligation clip creates two elongated points of tissue contact between each side of the inner surface of the first leg member and the inner surfaces of the elongated slot of the second leg member. The two elongated points of tissue contact provide improved sealing performance for the surgical ligation clip. The two elongated surfaces also provide a more even distribution of forces along the closed members and the tissue therein.

In certain embodiments of the surgical ligation clip, the proximal tissue shield extends inwardly from the inner surface of each side of the elongated slot on the proximal portion of the second leg member and past the tissue clamping inner surface of the first leg member when the leg members are in a partially closed position. The proximal tissue shield prevents tissue that is being clamped between the inner surfaces of the leg members from being pinched at the hinge.

In certain embodiments of the surgical ligation clip, the proximal tissue shield has a curved outer surface extending inwardly from the proximal portion of the second leg member and past the tissue clamping inner surface of the first leg member when the leg members are in a partially closed position. Thus, the proximal tissue shield prevents tissue that is being clamped between the inner surfaces of the leg members from being pinched and cut at the hinge to avoid undesired hemorrhaging, disassociating the hinge, or preventing closure of the ligation clip for implantation.

In certain embodiments of the surgical ligation clip, the proximal tissue shield extends inwardly from the inner surfaces of the first leg member and past the tissue clamping inner surface of the second leg member when the leg members are in a partially closed position. The proximal or distal tissue shields can be contoured in any shape (e.g., straight, curved, beveled) effective for moving tissue away from the hinge or locking mechanism portions of the clip intended to be shielded.

In certain embodiments of the surgical ligation clip, the inner surface of the first and second leg members have a convex radius of curvature with respect to each other between the proximal and distal ends. The convex bowing of the leg members maintains significant locking tension on the hook member and locking element when engaged. The invention contemplates that either or both leg members can alternatively be linear or have a concave radius.

In certain embodiments of the surgical ligation clip, the first and second leg members each have convex inner surfaces with respect to each other, such that the central portion of the first leg member extends through the elongated slot past the outer surface of the second leg member when the ligation clip is in the closed position and no tissue is present between the surfaces. This configuration provides the two elongated points of tissue contact to provide improved sealing performance for the ligation clip when tissue is present. This configuration also provides a dynamic force that permits the first and second legs to continuously bow inward as the tissue necroses.

In certain embodiments of the surgical ligation clip, the distal tissue shield extends along each side of the second leg member distally past the locking element. This configuration of the distal tissue shield provides protection in three directions (distally and along each side) for the locking means to effectively engage the hook member with minimal tissue interference.

In certain embodiments of the surgical ligation clip, the distal end of at least one leg member has a pair of opposing outer surface arcs for engaging concentric retaining arcs in jaws of an applicator tool, as described below. The opposing outer surface arcs on each distal end allow the ligation clips to securely and rotatably engage within the applicator jaws during closing and securing of the distal ends of the leg members together, and releasing the secured ligation clip, as described below.

In certain embodiments of the surgical ligation clip, the outer surface of the distal end of the first leg extends outwardly proximal to the hook member to form a first outer concentric arc and an opposing second inner concentric arc. In certain embodiments of the surgical ligation clip, the outer surface of the distal end of the second leg extends outwardly proximal to the locking element to form a first outer concentric arc and an opposing second inner concentric arc. The pair of opposing outer surface arcs on the distal ends of the leg members rotatably engage concentric retaining arcs in the jaws of an applicator tool. In certain embodiments of the surgical ligation clip, the ligation clip comprises both first and second opposing outer concentric arcs on each of the first and second leg members. In certain embodiments, the outer surface of the distal ends of the legs extend outwardly proximal to the hook member to form only one outer concentric arc on each leg to rotatably engage concentric retaining arcs in the jaws of an applicator tool.

In certain embodiments, the surgical ligation clip has an applicator advancement receptacle on the outer surface of the ligation clip adjacent to the hinge. The applicator advancement receptacle provides a point of contact for the ligation clip applicator advancement mechanism to move clips distally into position in the jaws of the applicator for delivery to patient tissue. In certain embodiments, the surgical ligation clip is sized and adapted for ligating pedicle tissue supporting a prostate gland.

The present invention further provides an applicator for storing, delivering and securing a surgical ligation clip. The applicator comprises an elongated chamber for receiving a plurality of surgical ligation clips individually arranged in line for indexed movement therethrough. The chamber of the applicator generally comprises a proximal end, a central clip storage region and an open distal end. The applicator for a surgical ligation clip further comprises a pair of jaws disposed on the open distal end of the chamber. Each ligation clip is temporarily retained in the applicator jaws for placement and closing engagement onto target tissue.

In certain embodiments, the surgical ligation clip applicator comprises a handle disposed on the proximal end of the elongated chamber for user engagement. The applicator further comprises a jaw actuator mechanism within the elongated chamber comprising an actuator rod in movable communication with the handle to open and close at least one of the jaws at the distal end of the apparatus. Various specific alternative configurations of actuator mechanisms will be apparent to one of skill in the art. In particular, the surgical clip actuator handle can be adapted for engagement with a robotic mechanism, rather than a human hand, for delivery of surgical ligation clips in robot-assisted procedures such as a robot assisted radical prostatectomy.

The applicator can further comprise a ligation clip advancement mechanism within the elongated chamber in movable communication with the handle for selectively indexing clips in line distally one at a time into the jaws. In certain embodiments, the ligation clip advancement mechanism within the elongated chamber comprises a movable arm with a pivoting clip ram at the distal end. The movable arm also has an indexing rack pivotally extending therefrom for contacting an applicator advancement receptacle on the outer surface of each ligation clip adjacent to the hinge. The proximal end of the arm is in communication with the handle for user engagement for forward clip movement and rearward indexing. Various specific alternative configurations of advancement and indexing mechanisms will be apparent to one of skill in the art, including again adaptation of the surgical clip advancement handle for engagement with a robotic mechanism, rather than a human hand, for delivery of surgical ligation clips in robot-assisted procedures.

In certain embodiments of the applicator for delivering and securing only one clip at a time, or more than one clip stacked side by side delivered and secured at one time, the applicator does not have a central clip storage region or a ligation clip advancement mechanism within the elongated chamber.

However, in certain embodiments where multiple clips are stored in the chamber, the clip applicator can further comprise a central rail in the elongated chamber upon which the surgical clips are directed. The ligation clips can ride on the central rail in contact with the inner surface of the second leg member. In some embodiments, the applicator central rail can also have back stops disposed at selected intervals along the length of the rail for prohibiting clips from moving proximally therepast. In some embodiments, two central rails are provided for carrying the second leg member, and a central trough is provided therebetween to accept the first leg member traveling distally along the length of the chamber.

In certain embodiments, at least one of the distal ends of the applicator jaws have a sharpened incising teeth member extending therefrom towards the other jaw. The jaws selectively articulate between an open position for retaining an open ligation clip therebetween, and a closed position for cutting, extracting, penetrating, eliminating, abstracting and/or removing ancillary tissue between opposing incising members to provide access to the target ligation tissue for securing the ligation clip in position.

In certain embodiments of the applicator for a surgical ligation clip, only one jaw articulates against the opposite fixed jaw. In certain embodiments, in the closed position the sharpened incising teeth member is located on the articulating jaw and moves in a shearing motion past each other jaw or teeth members when in the closed position. In certain embodiments, in the closed position the sharpened incising teeth members of the articulating jaw move proximally past the sharpened incising teeth members of the fixed jaw.

It is understood that the sharpened teeth members on each jaw can be of any comparative sharpness and in any relative shape or profile (e.g., straight, curved, sloped, serrated, etc.) effective for cutting tissue and in particular pedicle tissue. In some cases, the fixed jaw can be an unsharpened anvil for cutting thereon by a sharpened teeth member of the articulating jaw, or the fixed jaw can have an edge or slope for permitting the sharpened teeth member of the articulating jaw to move therepast. The sharpness and profile of teeth members can be determined by one of skill based on the intended tissue. By “sharpened” is meant that an edge has a smaller angle of protuberance than 45 degrees, 30 degrees, 20 degrees, 10 degrees, 5 degrees or smaller.

In certain embodiments of the surgical ligation clip applicator, the inner surfaces of the jaws have indentations and adjacent proximally extending retaining flanges for retaining the distal ends of the leg members of the ligation clip therein when the jaws are in the open position and releasing the leg members when the clip is in the closed position. In certain embodiments of the applicator, the inner surfaces of the jaws each have a pair of concentric retaining arcs and an adjacent proximally extending retaining flange for rotatably retaining a concentric pair of opposing outer surface arcs on distal ends of the leg members of the ligation clip therein when the jaws are in the open position and releasing the leg members when the clip is in the closed position. In this embodiment, each jaw of the applicator thus provides three retaining features (two concentric retaining arcs and a retaining flange) to securely position the ligation clip during tissue incising closure of the jaws and securing engagement of the hook member with the locking element.

In certain embodiments, the elongated chamber and jaws are selectively rotatable relative to the handle. Therefore, in embodiments where one jaw is movable and one jaw is fixed, either jaw can be positioned around the target tissue as desired in a 360 degree radius. This allows the user to optimize the angle of approach to the desired tissue depending upon the location and size of the incision.

In certain embodiments, the handle of the applicator comprises two separate user articulated elements for the jaw actuator mechanism and the ligation clip driving mechanism. This feature permits focusing more user generated mechanical leverage on each articulating movement, particularly advantageous when penetrating dense pedicle tissue. In alternative embodiments, the jaw actuator mechanism and the ligation clip driving mechanism can be manipulated by a single articulating movement.

The invention further provides methods of using the surgical ligation clips and applicator in a medical procedure. In certain embodiments, the medical procedure is ligation of pedicle tissues, and in particular pedicle tissues supporting a prostate gland. However, the various devices and systems described herein may be utilized as an accompaniment with any number of surgical procedures to ligate a variety of possible tissues, in addition to prostate pedicle tissue. For example, the improved ligation clips of the present invention are also particularly useful for ligation of uterine pedicle tissue during a hysterectomy and for ligation of bowel pedicle tissues during a resection procedure.

The invention further provides methods of manufacturing the surgical ligation clips and applicator such as would be apparent to one of skill in the art given the disclosure and objectives of the present invention. The surgical ligation clips and applicators can be constructed of any biocompatible, reliable, resilient or memory-shape materials, now known or later discovered in the future, including but not limited to polymers, copolymers, metals and metal alloys. Exemplary memory-shape material is a nickel-titanium alloy referred to as NITINOL, or a polyether ether ketone commonly known as PEEK. Thermosensitive materials which change resiliency at different temperatures are contemplated. The ligation clips can be constructed of any naturally occurring or synthetic, non-biodegradable, biodegradable or bioresorbable material. The ligation clips can be coated or impregnated with therapeutic agents, such as but not limited to, antiseptics, antibiotics, blood clotting factors, growth factors, or steroids. The ligation clips can be coated or impregnated with fluorescent or radiopaque materials for radiological imaging. The ligation clips can also be configured to be removable at the discretion of a physician.

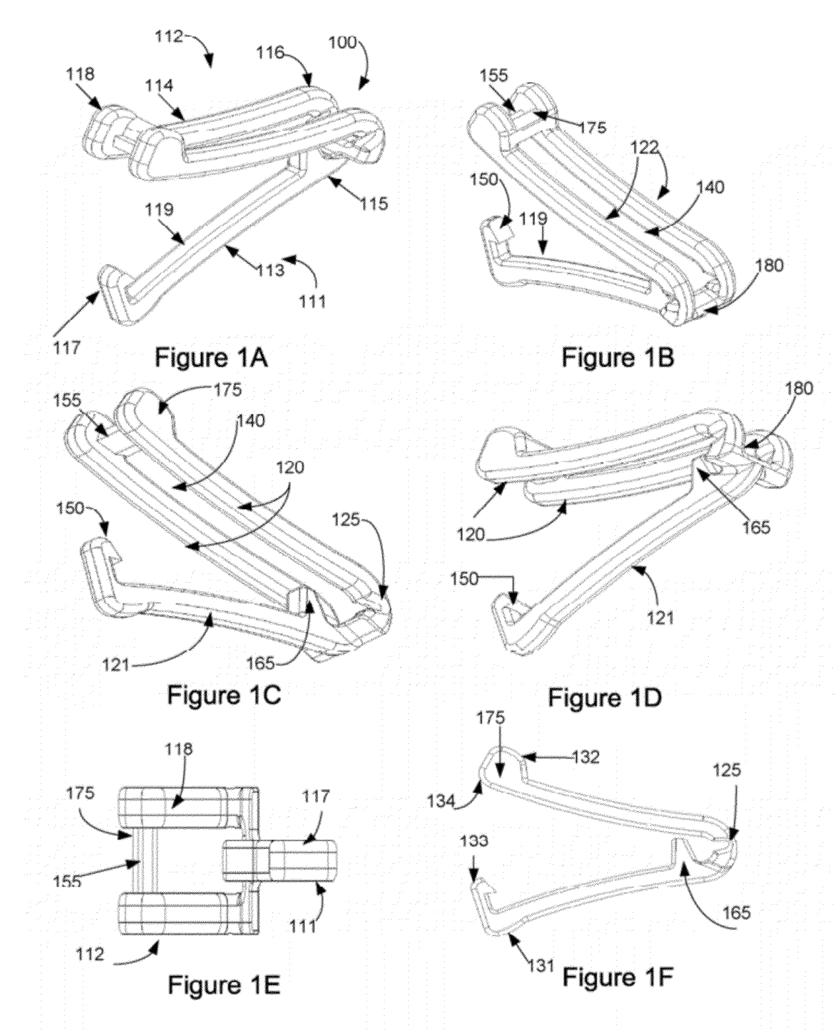

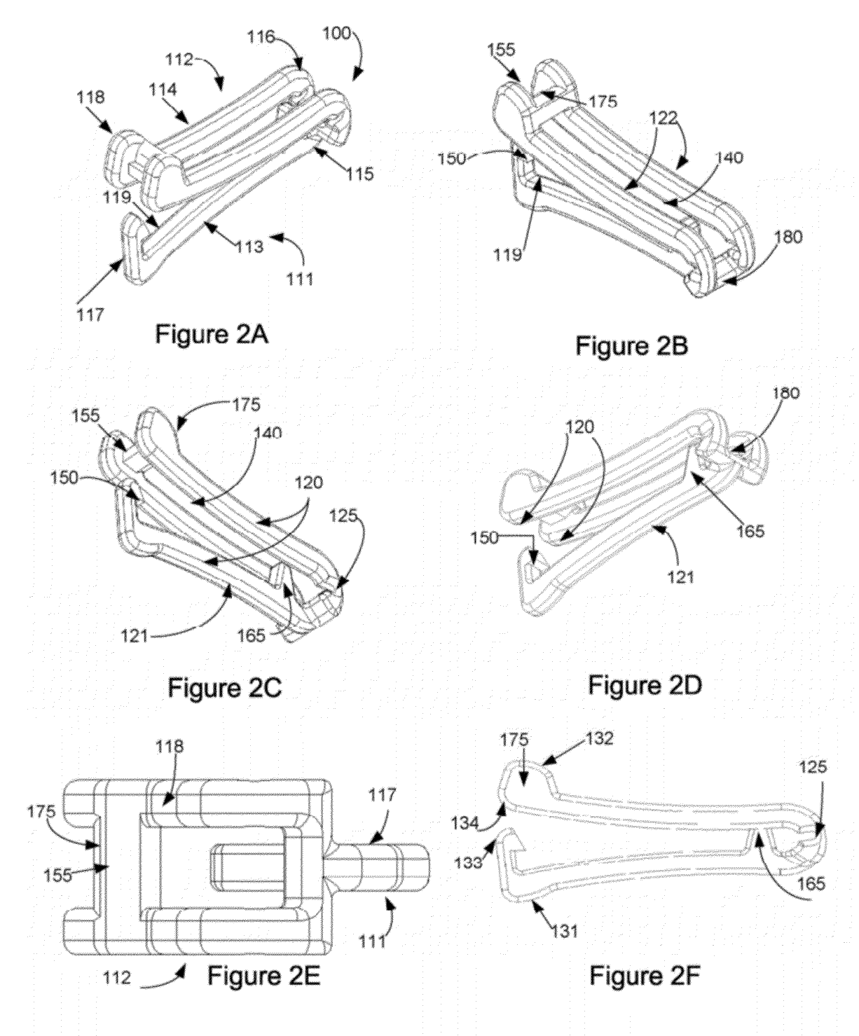

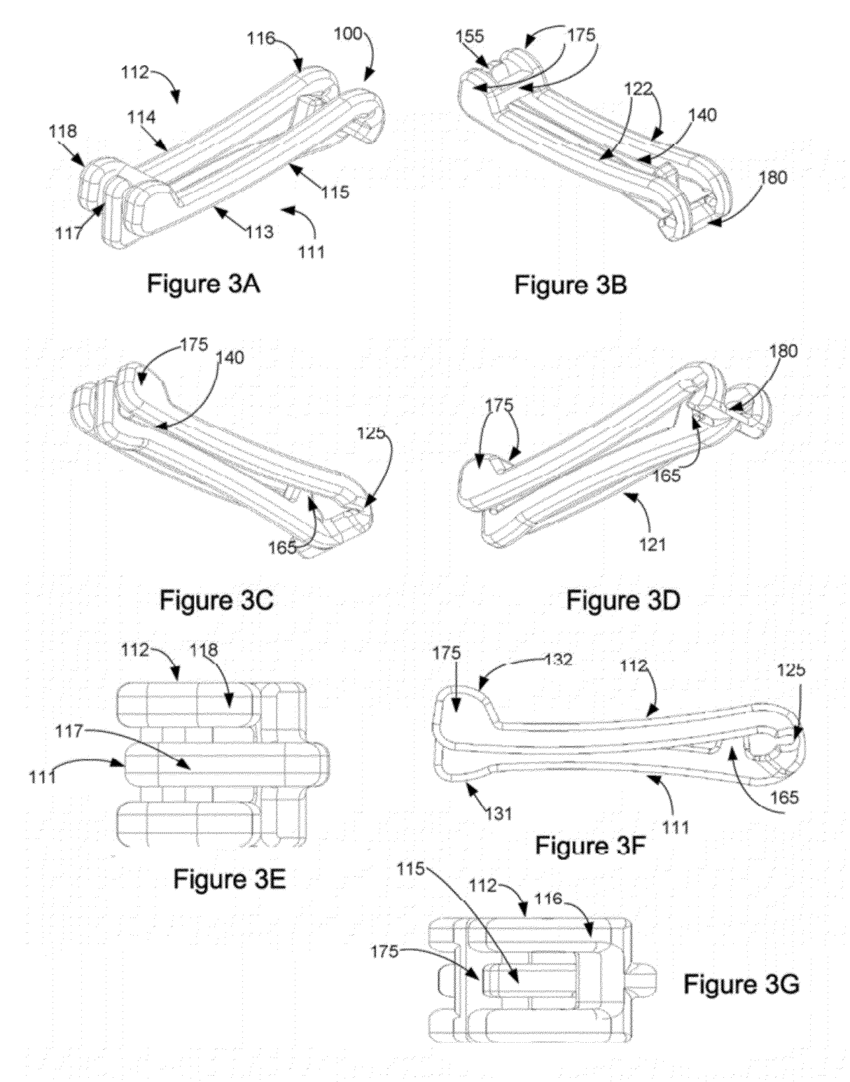

FIGS. 1A-1F illustrate various views of one embodiment of the present invention of a surgical tissue ligation clip 100 in the open position. FIGS. 2A-2F illustrate various views of the same embodiment of the present invention of a surgical tissue ligation clip 100 in a partially closed position. FIGS. 3A-3G illustrate various views of the same embodiment of the present invention of a surgical tissue ligation clip 100 in the closed position. The ligation clip 100 comprises first and second leg members 111, 112 each having a central portion 113, 114, a proximal end 115, 116, a distal end 117, 118, a tissue clamping inner surface 119, 120, and an opposite outer surface 121,122 with elongated sides, wherein the leg members 111, 112 are joined at their proximal ends 115, 116 by a resilient hinge 125 such that the tissue clamping inner surface of one leg member 119 is in movable opposition to the tissue clamping inner surface 120 of the other leg member 112.

The surgical tissue ligation clip 100 further comprises a deflectable hook member 150 on the distal end 117 of the first leg member 111 curved proximally toward said second leg member 112, and a locking element 155 on the distal end 118 of the second leg member 112 in a position complementary to said hook member 150, whereby when the first and second leg members 111, 112 are moved from an open position to a closed position about the hinge 125, the hook member 150 deflects about the locking element 155 to lock the clip 100 in a closed position.

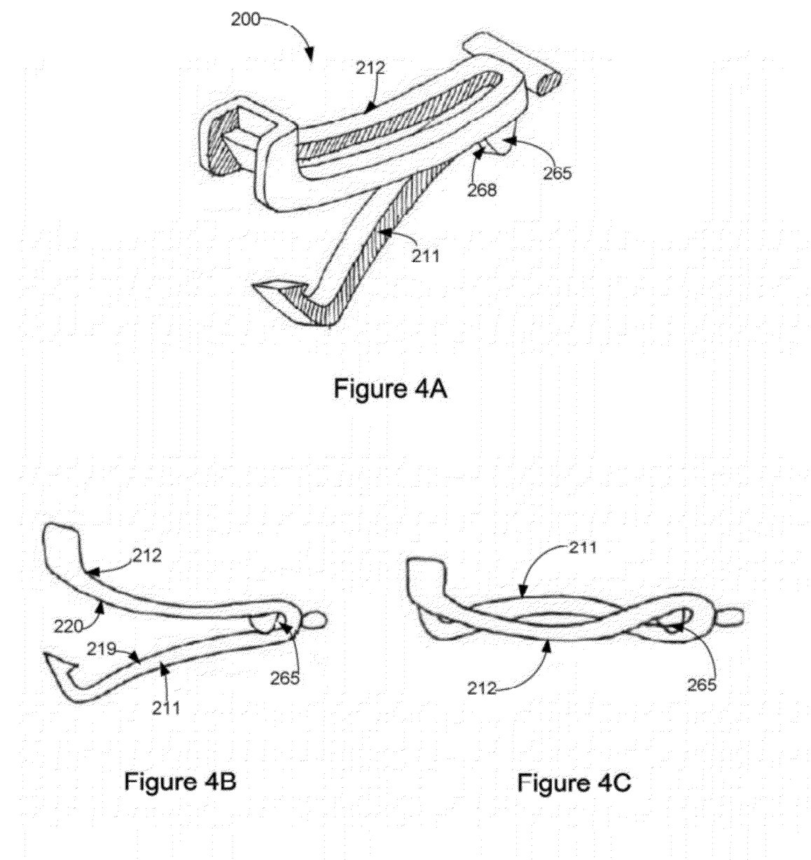

The surgical tissue ligation clip 100 further comprises a proximal tissue shield 165 extending inwardly from the proximal portion 115 of the first leg member 111 past the tissue clamping inner surface 120 of the opposite leg member 112 when the leg members 111, 112 are in partially closed and closed positions, wherein the proximal tissue shield 165 prevents tissue being clamped between the inner surfaces 119, 120 of the leg members 111, 112 from being pinched at the hinge 125. FIG. 4 shows an alternative embodiment of the surgical ligation clip, wherein the proximal tissue shield 165 extends from the proximal portion 116 of the second leg member 112, on each side past the tissue clamping inner surface 119 of the opposite leg member 111 when the leg members 111, 112 are in a partially closed position, such that the proximal tissue shield 165 prevents tissue being clamped between the inner surfaces of the leg members 111, 112 from being pinched at the hinge 125.

The surgical tissue ligation clip 100 can further comprise a distal tissue shield 175 extending outwardly from side to side of the distal end 118 of the outer surface 122 of the second leg member 112 proximal to locking element 155, wherein the distal tissue shield 175 prevents tissue being clamped between the inner surfaces 119, 120 of the leg members 111, 112 from being pinched between the hook member 150 and locking element 155.

As show in FIG.3 of the surgical ligation clip 100, the second leg member 112 defines a centrally located elongated slot 140 into which the central portion 113 of the first leg member 111 passes when the ligation clip 100 is in a closed position, and wherein the tissue clamping inner surface 120 of the second leg member 112 has two surfaces 120 which are in movable opposition to but not in contact with the inner surface 119 of the first leg member 111 when the ligation clip 100 is in a closed position. Therefore, as show in FIG. 3, closing the ligation clip 100 creates two elongated points of tissue contact between each side of the inner surface 119 of the first leg member 111 and the inner surfaces 120 of the elongated slot 140 of the second leg member 112.

As show in FIGS. 2 and 3 of the surgical ligation clip 100, the proximal tissue shield 165 extends inwardly from the inner surface 119, 120 of each side of the elongated slot 140 on the proximal end 116 of the second leg member 112 and past the tissue clamping inner surface 119 of the first leg member 111 when the leg members 111, 112 are in a partially closed and position, wherein the proximal tissue shield 165 prevents tissue being clamped between the inner surfaces 119, 120 of the leg members 111, 112 from being pinched at the hinge 125.

As shown in FIG. 4A-4C of an alternative embodiment of a tissue shield of the surgical ligation clip 200, the proximal tissue shield 265 has a curved outer surface 268 extending inwardly from the proximal portion 216 of the second leg member 212 and past the tissue clamping inner surface 219 of the first leg member 111 when the leg members 211, 212 are in a partially closed or position. The proximal tissue shield 265 can have two portions disposed on either side of the second leg member 212. The proximal tissue shield 265 prevents tissue being clamped between the inner surfaces 219, 220 of the leg members 211, 212 from being pinched at the hinge 225.

As show in FIGS. 1-3, in certain embodiments of the surgical ligation clip 100, the proximal tissue shield 165 extends inwardly from the inner surface 119 of the first leg member 111 and past the tissue clamping inner surface 120 of the second leg member 112 when the leg members 111, 112 are in a partially closed or position.

As show in FIGS. 1-3, in certain embodiments of the surgical ligation clip 100, the inner surfaces 119, 120 of the first and second leg members 111, 112 have a convex radius of curvature with respect to each other between the proximal ends 115, 116 and distal ends 117, 118.

Therefore, as shown in FIG. 3, in certain embodiments, the first and second leg members 111, 112 each have convex inner surfaces 119, 120 with respect to each other, such that the central portion 113 of the first leg member 111 extends through the elongated slot 140 past the, outer surface 120 of the second leg member 112 when the ligation clip 100 is in the closed position and no tissue is present between the surfaces 119, 120.

As show in FIGS. 1-3, in certain embodiments of the surgical ligation clip 100, the distal tissue shield 175 extends along each side of the second leg member 112 distally past the locking element 155, effectively shielding the hook member 150 and locking element 155 from tissue encroachment in three directions (proximally and on each side).

As show in FIGS. 1F, 2F and 3F, in certain embodiments of the surgical ligation clip 100, the distal end 117 of the first leg member 111 has a pair of opposing outer surface arcs 131, 133 for engaging concentric retaining arcs in jaws of an applicator tool, described in more detail below. The outer surface 121 of the distal end 117 of the first leg 111 extends outwardly proximal to the hook member 150 to form a first outer concentric arc 131 and an opposing second inner concentric arc 133.

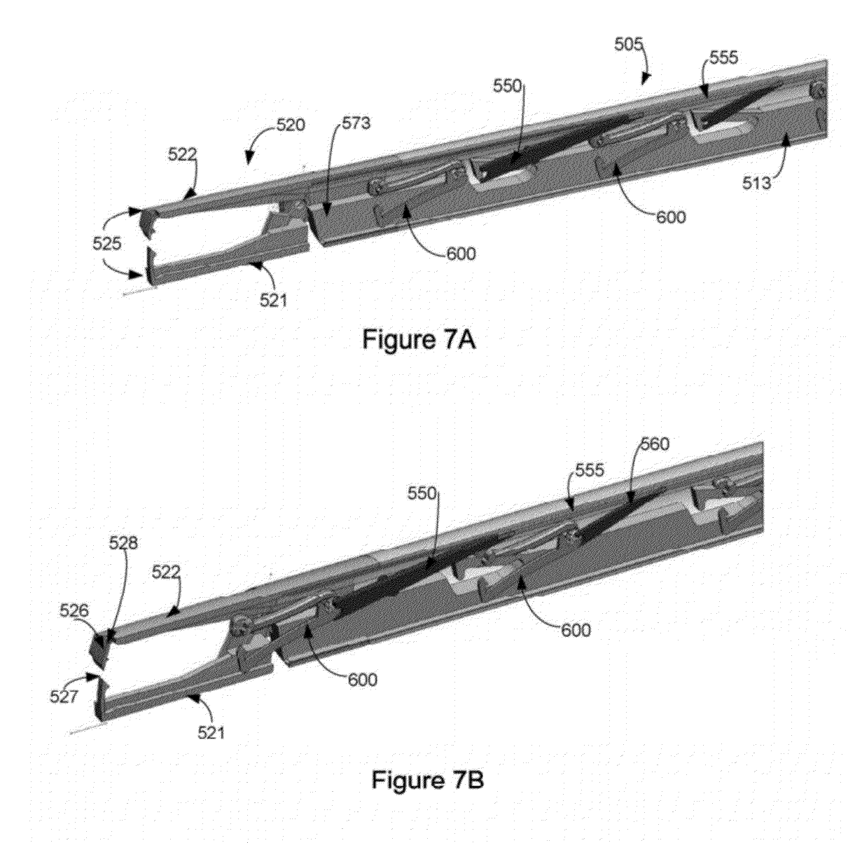

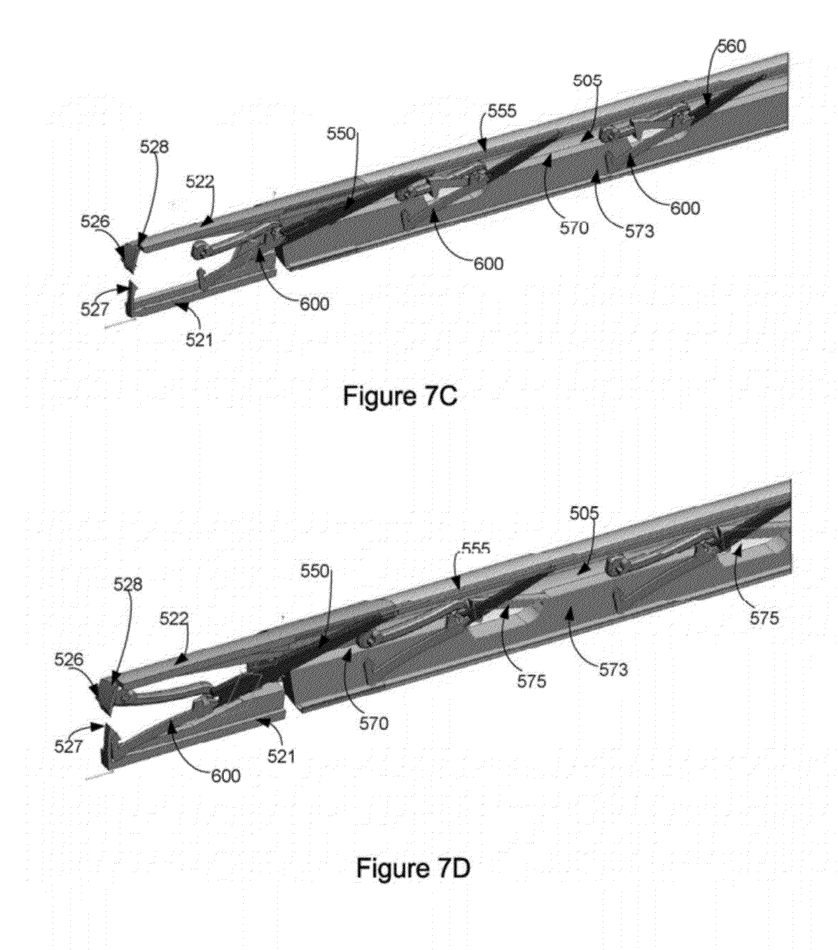

Furthermore, the distal end 118 of the second leg member 112 has a pair of opposing outer surface arcs 132, 134 for engaging concentric retaining arcs in jaws of an applicator tool. The outer surface 122 of the distal end 118 of the second leg 112 extends outwardly proximal to the locking element 155 to form a first outer concentric arc 132 and an opposing second inner concentric arc 134. Therefore, as shown, in certain embodiments, the surgical ligation clip 100 comprises both first 131, 132 and second 133, 134 opposing outer concentric arcs on each of the first and second leg members 111, 112. See also FIG. 7.

As show in FIGS. 1-3, in certain embodiments, the surgical ligation clip 100 has an applicator advancement receptacle 180 on the outer surface of the ligation clip 100 adjacent to the hinge 125. The applicator advancement receptacle 180 serves as a pivoting leverage point for moving the ligation clip 100 distally through the applicator.

In certain embodiments, the surgical ligation clip 100 is sized and adapted for ligating pedicle tissue supporting a prostate gland. In particular, in certain embodiments, the surgical ligation clip can be about 5 mm to 25 mm long, or about 14 mm long, and about 1 mm to 10 mm wide, or about 3 mm wide.

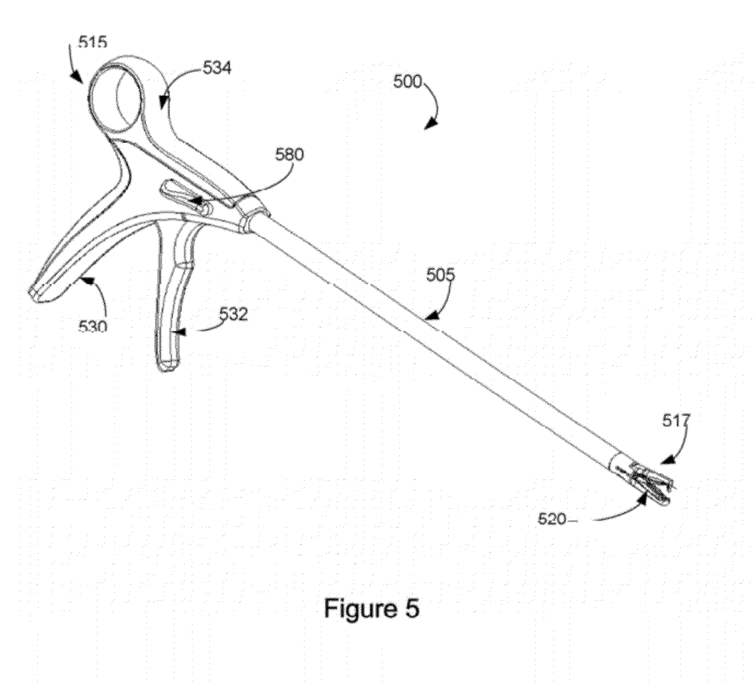

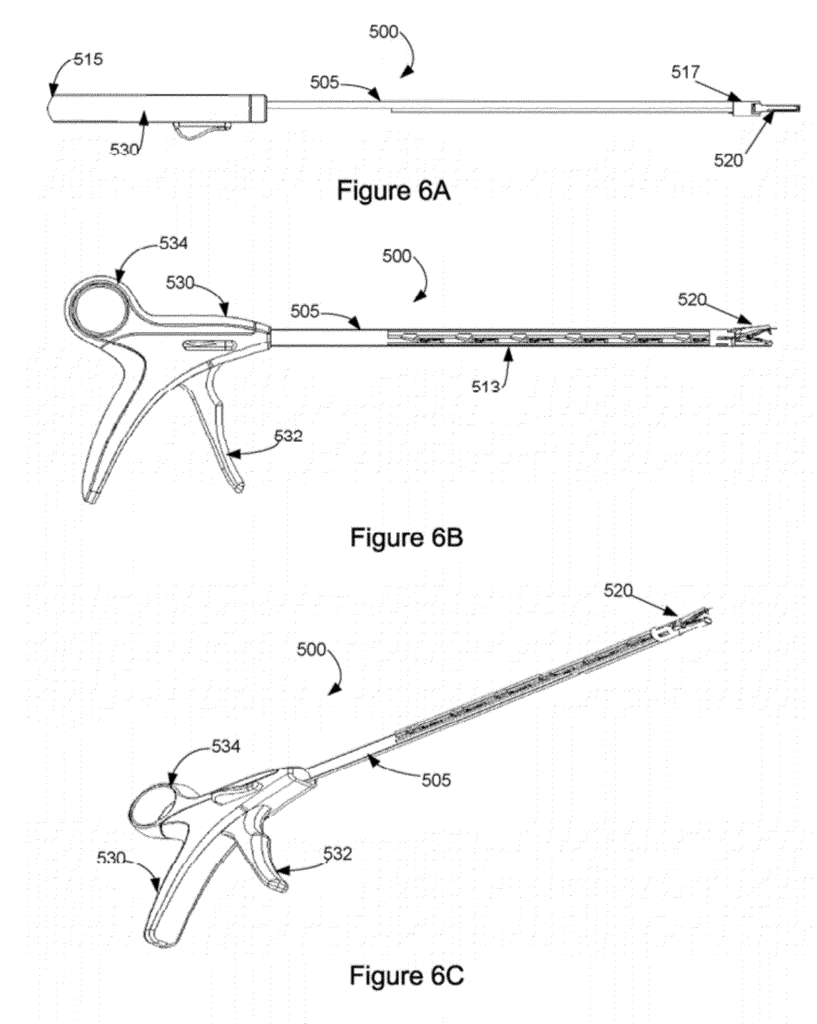

A illustrated in FIGS. 5-8, the present invention further provides an applicator 500 for a surgical ligation clip 600, comprising an elongated chamber 505 for receiving a plurality of surgical ligation clips individually arranged in line for movement therethrough. The chamber 505 has a proximal end 515, a central clip storage region 513, and an open distal end 517. The applicator 500 for a surgical ligation clip further comprises a pair of jaws 520 disposed on the open distal end of the chamber 505. The jaws 520 have an upper jaw element 521 and a lower jaw element 522. The jaws 520 have sharpened incising teeth members 525 extending towards each other for cutting tissue to access target ligation tissue. The jaws 520 selectively articulate between an open position for retaining an open ligation clip 600 therebetween, and a closed position for removing ancillary tissue between opposing incising teeth members 525 away from target tissue and securing the ligation clip 600 onto target tissue.

In certain embodiments, the surgical ligation clip applicator 500 comprises a handle 530 disposed on the proximal end 515 of the elongated chamber 505 for user engagement. The applicator 500 further comprises a jaw actuator mechanism within the elongated chamber 505 comprising a movable actuator rod 540 in responsive communication with the handle 530 to open and close at least one of the jaws elements 521, 522. The applicator 500 further comprises a jaw actuator mechanism within the elongated chamber 505 comprising a movable actuator rod 540 in responsive communication with the handle 530 to open and close at least one of the jaws elements 521, 522. As described above, the invention provides that surgical ligation clip applicator 500 can be configured for single clip delivery only without a chamber for storage of additional clips as shown in the drawings.

The applicator 500 as shown in the drawings further comprises a ligation clip advancement mechanism within the elongated chamber 505 in movable communication with the handle 530 for selectively indexing clips 600 distally one at a time into the jaws 520. In certain embodiments, the ligation clip advancement mechanism within the elongated chamber comprises a movable arm 555 with a distal pivoting clip ram 550 and an indexing rack 560 extending therefrom for contacting an applicator advancement receptacle 680 on the outer surface of the ligation clip 600 adjacent to the hinge 625. The proximal end of the arm 555 is in communication with a handle 534 for user engagement for distal (forward) clip movement and proximal (rearward) indexing of the rack 560.

The ring shown on handle 534 can be pulled proximally to move the advancement mechanism rearward for indexing the clip ram 550 and indexing rack 560 to engage new clips. The handle 534 is then pushed distally to move the advancement mechanism forward for advancing the clips and positioning the distal-most clip into the jaws 520. The applicator can be configured for storage and delivery of any number of clips, such as from 2 to 20 clips, or 7 clips. The applicator can also be equipped with an advancement mechanism lock 580 to prevent movement of the arm 555 during engagement of the clip leg members by closing the jaws 520. The advancement mechanism can be further provided with a forward biasing element, such as a spring, for maintaining distally directed pressure on the clip ram 550 during clip engagement with the jaws 520.

In certain embodiments, the applicator 500 further comprises a central rail 570 in the elongated chamber 505 upon which the surgical clips 600 ride, such as in contact with the inner tissue contacting surface 120 of the second leg member 612. The applicator 500 can further comprises a split central rail 570 in the elongated chamber 505 upon which each side of the second leg member 612 is carried, and a central trough 573 within which the first leg member 611 travels in alignment. In such embodiments, the applicator can also comprise back stops 575 disposed at selected intervals along the length of the rail 570 for prohibiting clips 600 from moving proximally, especially during rearward indexing of the advancement arm 555.

In certain embodiments of the applicator for a surgical ligation clip, only one jaw 521 articulates against the opposite fixed jaw 522. In certain embodiments, in the closed position the sharpened incising teeth members 525 move past each other in a shearing motion. In certain embodiments, in the closed position the sharpened incising teeth members 527 of the articulating jaw 521 move proximally past the sharpened incising teeth members 526 of the fixed jaw 522. In certain embodiments, the fixed jaw 522 has a gap 528 disposed therein proximal to the teeth members 526 in order to receive the reciprocating teeth members 527 on the movable jaw 521 to maximize the range of tissue incising action on the target tissue. The gap 528 further provides shearing forces on the proximal side of the teeth members 527 in addition to the distal shearing forces provided by the fixed teeth members 526. The combination of tissue cutting surfaces permits the ligation clip 600 to be securely closed without impinging on intact tissue.

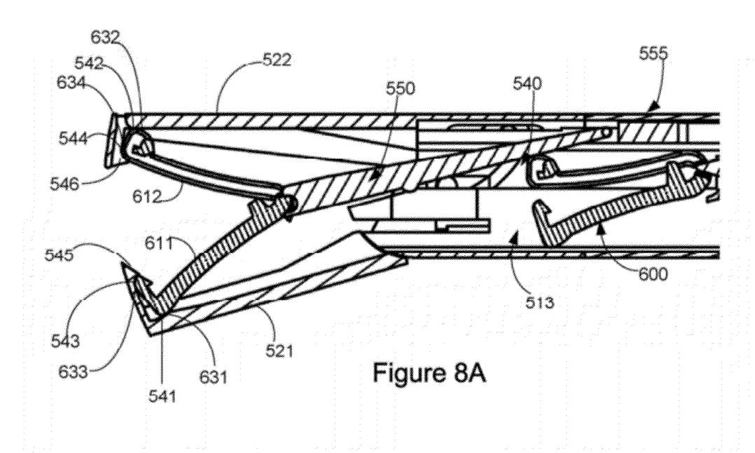

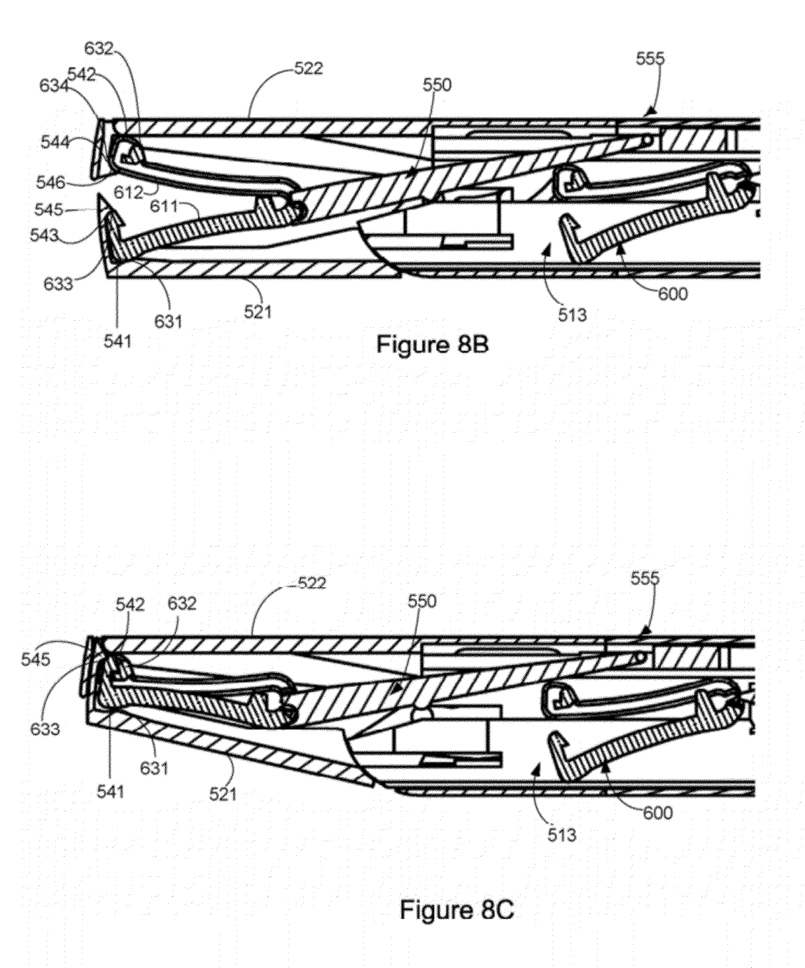

As can be seen in FIGS. 8A-8C, in certain embodiments of the applicator 500 for a surgical ligation clip, the inner surfaces of the fixed jaw 522 have indentations or retaining arcs 542, 544 and an adjacent proximally extending retaining flange 546 for retaining the distal end of the second leg member 612 of the ligation clip therein when the pair of jaws 520 are in the open position, and releasing the leg members 611, 612 when the clip is in the closed position. In certain embodiments, the retaining flange 546 on the fixed jaw can be bifurcated in order to allow the teeth members 527 on the articulating jaw 521 to extend through the gap 528. In certain embodiments of the applicator 500 for a surgical ligation clip, the inner surface of the movable jaw 521 has indentations or retaining arcs 541, 543 and an adjacent proximally extending retaining flange 545 for retaining the distal end of the first leg member 611 of the ligation clip 600 therein when the jaws 520 are in the open position and releasing the leg members 611, 612 when the clip 600 is in the closed position.

Therefore, in certain embodiments of the applicator, the inner surfaces of the jaws each have a pair of concentric retaining arcs and an adjacent proximally extending retaining flange for retaining a concentric pair of opposing outer surface arcs on distal ends of the leg members of the ligation clip therein when the jaws are in the open position and releasing the leg members when the clip is in the closed position. In this configuration of the applicator 500, the inner surfaces of the fixed jaw 522 have indentations or retaining arcs 542, 544 corresponding to concentric arcs 632, 634 respectively, on the second leg 612 of the ligation clip 600, in addition to an adjacent proximally extending retaining flange 546 for retaining the distal end of the second leg member 612 when the pair of jaws 520 are in the open and partially closed position, and releasing the leg members 612 when the clip is in the closed position. Likewise, in this configuration of the applicator 500, the inner surfaces of the movable jaw 521 has indentations or retaining arcs 541, 543 corresponding to concentric arcs 63I, 633 respectively, on the first leg 611 of the ligation clip 600, in addition to an adjacent proximally extending retaining flange 545 for retaining the distal end of the first leg member 611 of the ligation clip 600 therein when the jaws 520 are in the open and partially closed position, and then releasing the leg members 611, 612 when the clip 600 is in the closed position. This three point retaining system on each jaw 521, 522 provides a secure pivoting movement throughout closing of the ligation clip, and release of the ligation clip 600 from the jaws 521, 522 in the closed position.

For example, as can be seen in FIGS. 8A-8C, the opposing retaining arcs 631, 633 on distal end of the leg member 621 of the ligation clip 600 rotate through a bearing configuration against the two concentric retaining arcs 541, 543 and the adjacent proximally extending retaining flange 545 on the movable jaw 521 to keep the clip in position within the jaws when the jaws are in the open position of FIG. 8A and throughout the tissue incising progression of FIG. 8B. When the jaws 521, 522 and clip 600 are closed in FIG. 8C, with the locking mechanism engaged, the opposing bearing and retaining forces are reduced to permit release of the leg member 621 and clip onto the tissue as the jaws 521, 522 are re-opened. It is understood that each jaw can alternatively have any combination of one or two concentric retaining arcs and/or an adjacent proximally extending retaining flange, and the ligation clip can have one or more corresponding retaining arcs on distal ends of the leg members to keep the clip in position in the jaws when the jaws are in the open position and incising, and then reduce the retaining forces for release of each leg member when the clip is in the closed position.

Many modifications and other embodiments of the invention will come to mind to one skilled in the art to which this invention pertains and having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A surgical tissue ligation clip, comprising
a. first and second leg members each having a central portion, a proximal end, a distal end, a tissue clamping inner surface, and an opposite outer surface with elongated sides, wherein the leg members are joined at their proximal ends by a resilient hinge such that the tissue clamping inner surface of one leg member is in movable opposition to the tissue clamping inner surface of the other leg member,
b. a deflectable hook member on the distal end of the first leg member curved proximally toward said second leg member, and a locking element on the distal end of the second leg member in a position complementary to said hook member, whereby when the first and second leg members are moved from an open position to a closed position about the hinge, the hook member deflects about the locking element to lock the clip in a closed position,
c. a proximal tissue shield extending inwardly from the proximal end of the first or second leg members past the tissue clamping inner surface of the opposite leg member when the leg members are in a partially closed position, wherein the proximal tissue shield prevents tissue being clamped between the inner surfaces of the leg members from being pinched at the hinge, and
d. a distal tissue shield extending outwardly from side to side of the distal portion of the outer surface of the second leg member proximal to locking element, wherein the distal tissue shield prevents tissue being clamped between the inner surfaces of the leg members from being pinched between the hook member and locking element.

2. The surgical ligation clip of claim 1, wherein the second leg member defines a centrally located elongated slot into which the central portion of the first leg member passes when the ligation clip is in a closed position, and wherein the tissue clamping inner surface of the second leg member has two surfaces which are in movable opposition to but not in contact with the inner surface of the first leg member when the ligation clip is in a closed position.

3. The surgical ligation clip of claim 2, wherein closing the ligation clip creates two elongated points of tissue contact between each side of the inner surface of the first leg member and the inner surfaces of the elongated slot of the second leg member.

4. The surgical ligation clip of claim 3, wherein the proximal tissue shield extends inwardly from the inner surface of each side of the elongated slot on the proximal portion of the second leg member and past the tissue clamping inner surface of the first leg member when the leg members are in a partially closed position, wherein the proximal tissue shield prevents tissue being clamped between the inner surfaces of the leg members from being pinched at the hinge.

5. The surgical ligation clip of claim 3, wherein the proximal tissue shield has a curved outer surface extending inwardly from the proximal portion of the second leg member and past the tissue clamping inner surface of the first leg member when the leg members are in a partially closed position, wherein the proximal tissue shield prevents tissue being clamped between the inner surfaces of the leg members from being pinched at the hinge.

6. The surgical ligation clip of claim 3, wherein the proximal tissue shield extends inwardly from the inner surfaces of the first leg member and past the tissue clamping inner surface of the second leg member when the leg members are in a partially closed position.

7. The surgical ligation clip of claim 1, wherein the inner surface of the first and second leg members have a convex radius of curvature with respect to each other between the proximal and distal ends.

8. The surgical ligation clip of claim 7, wherein the first and second leg members each have convex inner surfaces with respect to each other, such that the central portion of the first leg member extends through the elongated slot past the outer surface of the second leg member when the ligation clip is in the closed position and no tissue is present between the surfaces.

9. The surgical ligation clip of claim 1, wherein the distal tissue shield extends along each side of the second leg member distally past the locking element.

10. The surgical ligation clip of claim 1, wherein the distal end of at least one leg members has a pair of opposing outer surface arcs for engaging concentric retaining arcs in jaws of an applicator tool.

11. The surgical ligation clip of claim 1, wherein the outer surface of the distal end of the first leg extends outwardly proximal to the hook member to form a first outer concentric arc and an opposing second inner concentric arc.

12. The surgical ligation clip of claim 11, wherein the outer surface of the distal end of the second leg extends outwardly proximal to the locking element to form a first outer concentric arc and an opposing second inner concentric arc.

13. The surgical ligation clip of claim 12, wherein the ligation clip comprises both first and second opposing outer concentric arcs on each of the first and second leg members.

14. The surgical ligation clip of claim 1, further comprising an applicator advancement receptacle on the outer surface of the ligation clip adjacent to the hinge.

15. An applicator for a surgical ligation clip, comprising
a. an elongated chamber comprising a proximal end, a central region and an open distal end,
b. a pair of jaws disposed on the open distal end of the chamber, at least one jaw having a sharpened incising teeth member extending therefrom towards the other jaw, wherein the jaws selectively articulate between an open position for retaining an open ligation clip therebetween and a closed position for removing ancillary tissue with the incising teeth member away from target tissue and securing the ligation clip onto target tissue,
c. a handle disposed on the proximal end of the elongated chamber for user engagement, and
d. a jaw actuator mechanism within the elongated chamber comprising a movable actuator rod in movable communication with the handle to open and close at least one of the jaws.

16. The applicator for a surgical ligation clip of claim 15, wherein the elongated chamber is configured for receiving a plurality of surgical ligation clips individually arranged in line for movement therethrough, and further comprising a ligation clip advancement mechanism within the elongated chamber in movable communication with the handle for selectively indexing clips distally one at a time into the jaws.

17. The applicator for a surgical ligation clip of claim 16, wherein the ligation clip advancement mechanism within the elongated chamber comprises a movable arm with and an indexing rack extending therefrom for contacting an applicator advancement receptacle on the outer surface of the ligation clip adjacent to the hinge, wherein the proximal end of the arm is in communication with a handle for user engagement for forward clip movement and rearward indexing.

18. The applicator for a surgical ligation clip of claim 16, further comprising a central rail in the elongated chamber upon which the surgical clips ride in contact with the inner surface of the second leg member, and comprising back stops disposed at selected intervals along the length of the rail for prohibiting clips from moving proximally therepast.

19. The applicator for a surgical ligation clip of claim 15, wherein only one jaw articulates against the opposite fixed jaw.

20. The applicator for a surgical ligation clip of claim 19, wherein both jaws have sharpened incising teeth members.

21. The applicator for a surgical ligation clip of claim 19, wherein in the closed position the sharpened incising teeth member moves past distal ends of the opposing jaw when in the closed position.

22. The applicator for a surgical ligation clip of claim 20, wherein in the closed position the sharpened incising teeth members of the articulating jaw move proximally past the sharpened incising teeth members of the fixed jaw.

23. The applicator for a surgical ligation clip of claim 15, wherein the inner surfaces of the jaws each have a pair of concentric retaining arcs and an adjacent proximally extending retaining flange for retaining a concentric pair of opposing outer surface arcs on distal ends of the leg members of the ligation clip therein when the jaws are in the open position and releasing the leg members when the clip is in the closed position.

24. The applicator for a surgical ligation clip of claim 15, wherein the elongated chamber and jaws are selectively rotatable relative to the handle.

25. The applicator for a surgical ligation clip of claim 16, wherein the handle comprises two separate user manipulable elements for the jaw actuator mechanism and the ligation clip driving mechanism.

26. (canceled)
